(19) United States
(12) Patent Application Publication
(10) Pub. No.: US 2019/0246729 A1
(43) Pub. Date: Aug. 15, 2019

(54) MAGNETIC FALSE EYELASHES AND EYEBROWS

(71) Applicant: Paris Presents Incorporated, Gurnee, IL (US)

(72) Inventor: Alyssa C. Nicoline, McHenry, IL (US)

(21) Appl. No.: 15/896,627

(22) Filed: Feb. 14, 2018

(51) Int. Cl. A41G 5/02; A61K 8/19; A61Q 1/00; A61Q 1/10

(52) U.S. Cl. CPC A41G 5/02; A61K 8/19; A45D 40/20; A61Q 1/10; A45D 2200/25; A61Q 1/00

(57) ABSTRACT

An exemplary method of applying a false eyelash or eyebrow hair comprises the steps of applying a cosmetic product to a natural eyelash, a natural eyebrow, an area around the natural eyelash, or an area around the natural eyebrow, the cosmetic product comprising magnetic properties and applying a false eyelash or eyebrow hair to the cosmetic product, at least a portion of the false eyelash or eyebrow hair comprising magnetic properties.

### TECHNICAL FIELD

The embodiments described herein relate to false eyelashes and eyebrows and, more particularly, to magnetic false eyelash and eyebrow hairs that adhere to natural eyelashes and eyebrows through various cosmetic products with magnetic formulas.

### BACKGROUND OF THE INVENTION

People are often dissatisfied with the length and thickness of their eyelashes and/or eyebrows. False eyelashes historically have been used by people who lack thick, long lashes, or people who prefer an appearance of thicker, longer lashes that mascara alone cannot provide. Previous false eyelashes include both strips of lashes that cover an entire lash line and individual eyelashes that are applied at different locations along the lash line. False eyelashes have traditionally been applied close to the lash line on the eyelid by an adhesive glue and generally include synthetic, human, or animal hairs attached to an elongated substrate, which may comprise a textile or non-woven material with a skin-facing surface and an outward-facing surface. The substrate attaches to the eyelid of a user by application of an adhesive either to the skin-facing surface or directly to the eyelid of the user. Such application suffers from a number of disadvantages.

First, the above-described false eyelashes are generally single-use items. The application of adhesive glue typically deforms the shape of false eyelash strips, either through application, wear, or removal. The removal process can deform the shape of false eyelash strips and, even with individual false eyelashes, the dried adhesive often clumps to the end of the lash and makes re-use difficult. The adhesive also frequently makes unwanted contact with the lashes themselves, causing the lashes to stick together and both impair the overall appearance and prevent re-use.

Further, the application of prior art false eyelashes may result in various user discomforts. For example, users often find that removal of the lashes and of the adhesive rips out natural eyelashes since the adhesive inevitably makes contact with the natural eyelashes of the user. The same result also occurs if the false eyelashes become caught on fibers from clothing or other material. Additionally, the substrates are often too long or short for the eyelid shape of the user, which can cause discomfort, especially after the addition of a thick adhesive glue. The adhesive glue is also prone to clumping, which may cause discomfort and impair the overall appearance of the false eyelashes. Some users also report sensitivity to the adhesive glue, complaining of eye irritation and difficult-to-remove residue left on the eyelid.

Still further, many adhesive glue formulations are broken down by the use of mascara or other cosmetic products, including most makeup removers. For example, when the adhesive glue is in close contact or mixes with certain types of mascara or other cosmetic products, the adhesive dissolves faster, causing the false eyelashes to separate from the lid and impair the overall appearance. Should the adhesive come into contact with most makeup removers, it can lose its adhesive qualities, causing the false eyelashes to separate from the natural lashes and deforming the structure of the substrate. This effect makes it difficult for the false eyelashes to remain intact or in good condition after removing eye makeup.

A relatively new alternative to adhesive false eyelashes is the non-adhesive false eyelash strip consisting of upper and lower eyelash assemblies that secure through magnetic attraction to the natural eyelash, which is sandwiched therebetween. While this method lacks the problems associated with adhesive glue, the method still suffers from a number of disadvantages. Namely, most magnetic strips only cover the center portion of a false eyelash strip, resulting in the appearance of unattached eyelashes along the outer edges. Users also report difficulty in placing the magnets close enough to the lash line to create a natural, attached appearance. Alternatively, users may experience pain and discomfort from pinching the eyelid with the magnets while attempting to clip the magnets too close to the eyelid. Because the magnets make up an entire strip attached to the false eyelash strip substrate, the result is a heavier, thicker, more noticeable product.

Likewise, many people look to false eyebrows to create the appearance of natural, full eyebrows that cosmetic products such as pencils and eyebrow mascara cannot alone achieve. Particularly in recent years, fashion and beauty trends have emphasized thick, full eyebrows as a beauty standard, making false eyebrows a much more sought-after beauty product. However, false eyebrows in the prior art generally consist of mere eyebrow extensions or “stick on eyebrow wigs.” Eyebrow extensions are generally created by a cosmetologist professional who glues individual synthetic, human, or animal hairs to existing eyebrow hairs of a user. The eyebrow extensions suffer from a number of disadvantages, including one-time use that lasts only several days to several weeks. These applications are also typically expensive and users run the risk of losing their natural brows as the false individual hairs fall off or are pulled out.

“Stick on eyebrow wigs,” in contrast, are full eyebrow applications generally marketed toward people with thinning or bald eyebrows. Such full false eyebrow applications have historically been applied directly to bald or nearly hairless skin over the brow bone with adhesive glue. Like false eyelashes, false eyebrow application strips may consist of synthetic, human, or animal hairs attached to an elongated substrate. That substrate may be a textile or non-woven material with a skin-facing surface and an outward-facing surface upon which the false eyebrow hairs extend primarily laterally toward an outer corner of the substrate, in relation to the face of the user. The substrate attaches to a brow bone of a user by application of an adhesive either to the skin-facing surface or directly to the brow bone of the user or by a peel-away adhesive on the skin-facing surface. Such false eyebrows suffer from a number of disadvantages. One primary disadvantage is the failure of appearing authentic. For example, the substrates beneath the hair are often either not the correct skin tone for the user, or if translucent, possess noticeable, unnatural shine. They also often do not sufficiently move and flex with the user's face for different facial expressions, making them unnatural and noticeable.

### SUMMARY

According to one aspect, a method of applying a false eyelash or eyebrow hair comprises the steps of applying a cosmetic product to a natural eyelash, a natural eyebrow, an area around the natural eyelash, or an area around the natural eyebrow, the cosmetic product comprising magnetic properties and applying a false eyelash or eyebrow hair to the cosmetic product, at least a portion of the false eyelash or eyebrow hair comprising magnetic properties.

According to another aspect, a system for applying a hair comprises a cosmetic product comprising magnetic properties and a false hair comprising magnetic properties.

According to a further aspect, a kit for applying a false eyelash or eyebrow hair comprises a cosmetic product comprising magnetic properties, an applicator for applying the cosmetic product, and a false eyelash or eyebrow hair comprising magnetic properties.

### BRIEF DESCRIPTION OF THE DRAWINGS

FIGS. 1A and 1B depict methods for applying a false eyelash system;

FIG. 2 depicts a method for applying a false eyebrow system;

FIGS. 3A-3D and 4A-4D depict exemplary cosmetic products that may contain a magnetic formula used to attract magnetically treated false eyelashes or false eyebrows;

FIGS. 5A, 5B and 5C depict various methods for infusing or coating a human or animal hair or synthetic fiber with a magnetic formula;

FIGS. 6A-6H depict the application of a cosmetic product with magnetic formula to eyelashes and eyelids of a user;

FIGS. 7A-7H depict the application of a cosmetic product with magnetic formula to eyebrows and brow bone skin of a user;

FIG. 8 depicts the application of a full, false eyebrow to a natural eyebrow or brow bone area of a user; and

FIGS. 9 and 10 depict various methods for removing magnetically applied false eyelashes and eyebrows.

### DETAILED DESCRIPTION OF THE INVENTION

It is an object of the present disclosure to overcome the disadvantages of existing false eyelashes, false eyebrows, and false eyelash and eyebrow systems by providing magnetic false eyelash and eyebrow solutions that can be easily and comfortably reused. It is a further object of the present disclosure to provide options for magnetically applying false eyelash hairs and false eyebrow hairs while incorporating a cosmetic element.

These and other objects are achieved through the false eyelash and eyebrow systems of the present disclosure as described hereinafter.

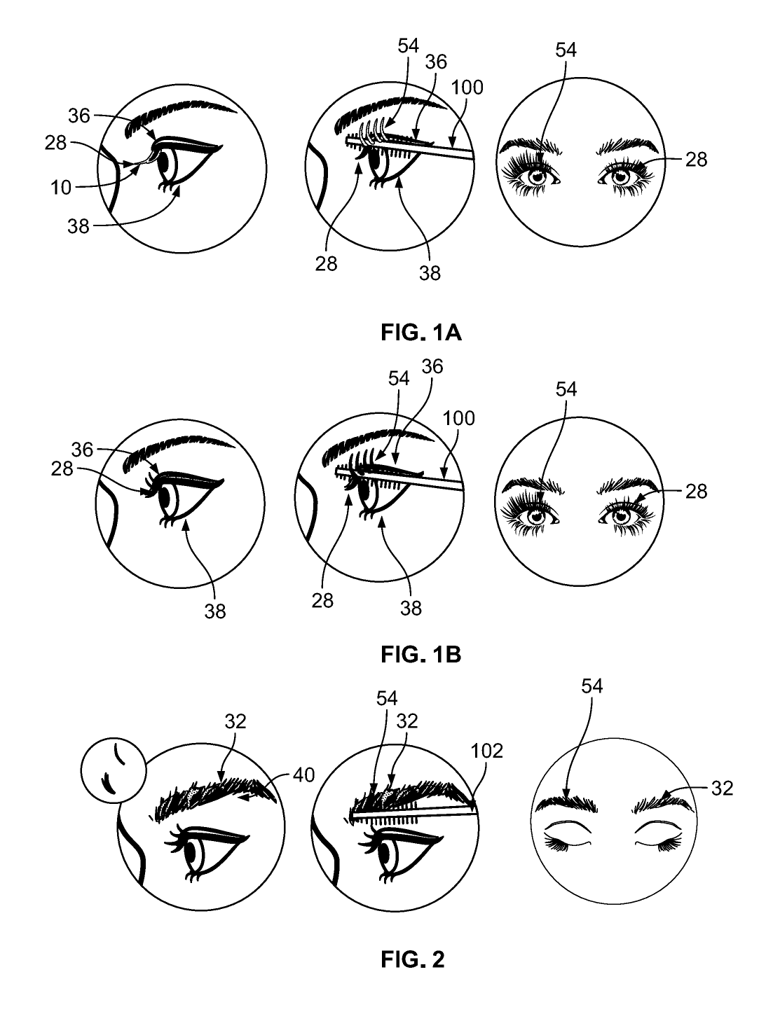

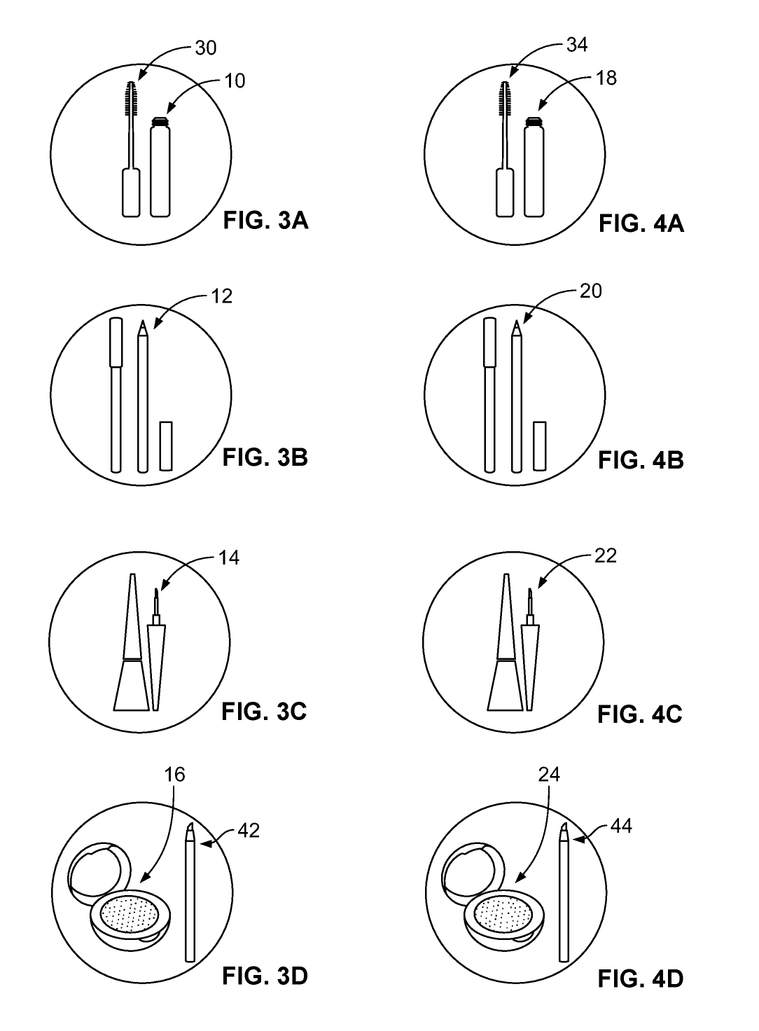

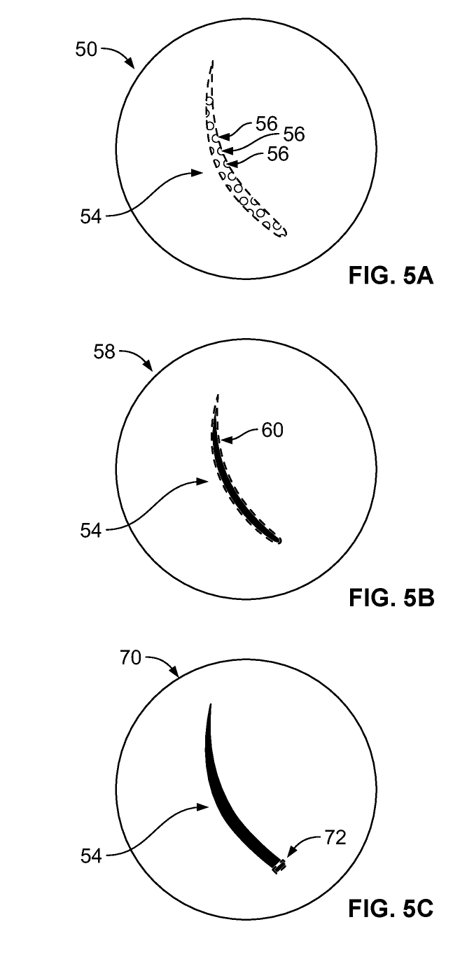

The false eyelash system, as seen in FIG. 1, and the false eyebrow system, as seen in FIG. 2, of the present disclosure comprise two primary components, both of which may take several forms. The first component, as seen in FIGS. 3 and 4, is a formula having magnetic properties, the formula being present in a variety of cosmetic products to be applied to the eyelid, eyelashes, and/or brow area of a user. The second component, as seen in FIG. 5, is a false eyelash or eyebrow hair, which may be an individual, a cluster, or a strip of synthetic fiber(s) and/or human or animal hair(s) that have magnetic properties. The general reference herein to something having magnetic properties or being magnetic or magnetized (or use of magnetization) means that element includes a material that exhibits properties of magnetism or is attracted to a material that exhibits properties of magnetism. The magnetic properties of the magnetic formula within the cosmetic products and the false eyelash and eyebrow hairs described herein may be achieved in a variety of different manners, as discussed in more detail below.

The first component, the magnetic formula, may be formed to include any suitable magnetic properties, for example, the magnetic formula may include a material that exhibits properties of magnetism or may include one or more of iron, magnesium, cobalt, nickel, or any other magnetic metal in any form, whether liquid, solid, gel, foam, solid, or any other suitable form. The magnetic formula may be present in and may be applied through cosmetic products including, but not limited to, mascara 10, gel or cream eyeliner pencils 12, liquid or cream eyeliners 14, eyeshadow powders 16, eyebrow mascaras 18, gel or cream eyebrow pencils 20, liquid or cream eyebrow pencils 22, and eyebrow powders 24. Most cosmetic products including the magnetic formula will have other components, for example, that provide aesthetic or other properties. The magnetic formula itself may therefore be translucent. In other embodiments, the magnetic formula may produce colors including but not limited to black, brown, gray, gold, silver, bronze, white, tan, purple, green, blue, red, yellow, orange, etc. Beyond the magnetic properties of the magnetic formula within the cosmetic products, the cosmetic products respectively operate as standard cosmetic products, for example, as standard eyelash or eyebrow mascara 10, 18, eye pencils and/or liners 12, 14, eyebrow pencils and/or liners 20, 22, and eyeshadow or eyebrow powder 16, 24. As such, users achieve the benefit of a makeup product that doubles as the source of attachment for comfortable, reusable false eyelashes and/or eyebrows. While various types of cosmetic products are shown and described herein, it is contemplated that the magnetic formula of the present invention may be utilized with any cosmetic product.

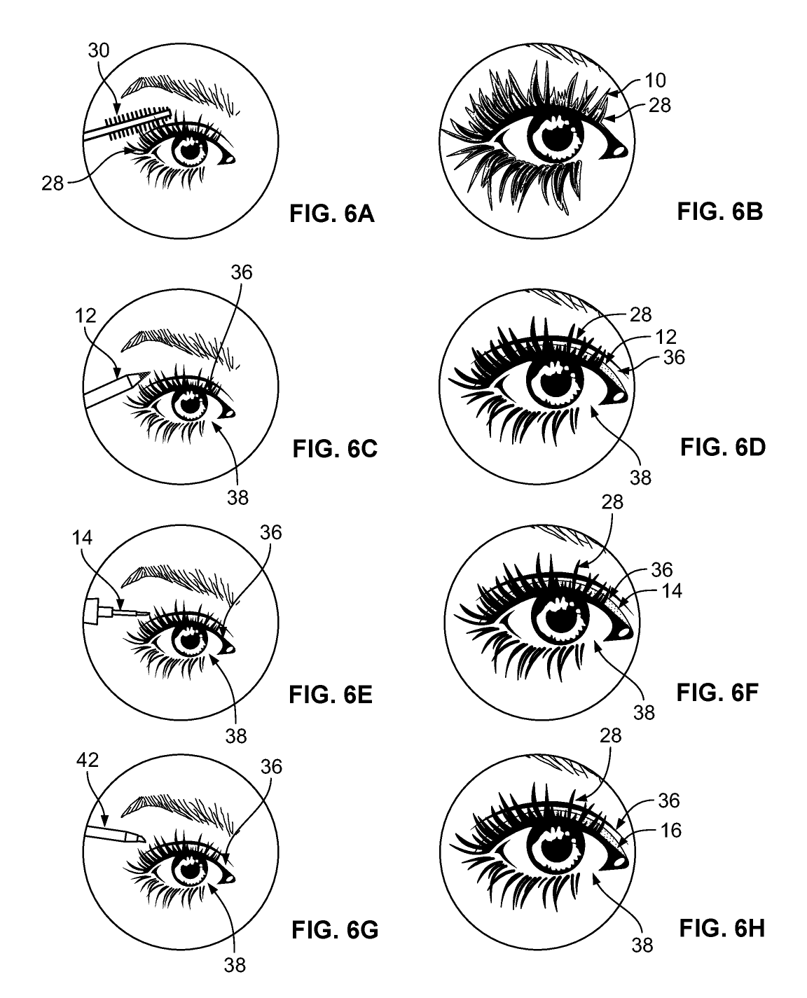

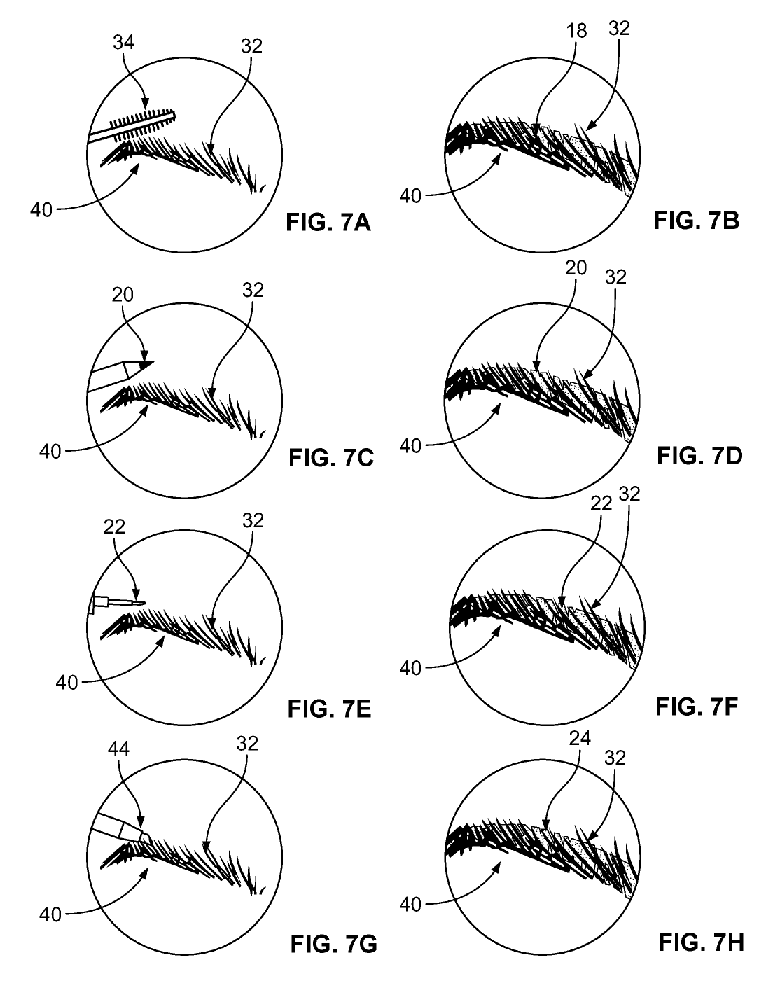

As seen in FIGS. 6 and 7, the magnetic formula may be used as a component of, for example, eyelash mascara 10, which may be applied directly to the natural eyelashes 28 of a user using a wand, comb, or other applicator 30, or eyebrow mascara 18, which is applied directly to the natural eyebrows 32 of a user using a wand, comb, or other applicator 34. Such application may be done in short strokes or long strokes to spread the mascara 10, 18 including the magnetic formula along the natural hairs meant to be supplemented by connection of false, magnetic hairs, as will be discussed in more detail hereinafter. The magnetic formula may also be used in conjunction with gel, cream, liquid eyeliner pencils 12, 14 or any other suitable cosmetic products, which are applied directly to an upper and/or lower eyelid 36, 38 of a user, or with gel, cream, or liquid eyebrow pencils 20, 22 which are applied directly to natural eyebrows 32 and/or brow bone skin 40 of a user. On both the eyelids 36, 38 and the eyebrows and brow bone skin 32, 40, the penciling and/or lining may be done in short strokes, long strokes, or dots; there should simply be sufficient lining where the false hairs are meant to connect and protrude. In other embodiments, the magnetic formula may also be used as a component of eyeshadow powder 16, which is applied directly to the eyelids 36, 38 of a user using a brush or other applicator 42, or with eyebrow powder 24, which is applied directly to natural eyebrows 32 and/or brow bone skin 40 of a user using a brush or other applicator 44. On both the eyelids 36, 38 and the eyebrows and brow bone skin 32, 40, the powder may be applied in short strokes, long strokes, or dots; there must simply be sufficient powder where the magnetic hairs are meant to connect and protrude.

As noted above, FIG. 5 shows the second component of the false eyelash and eyebrow systems of FIGS. 1 and 2, which are the false eyelash and eyebrow hairs 54 with magnetic properties. The false eyelash and/or eyebrow hairs 54 may include a material that exhibits properties of magnetism or may include one or more of iron, magnesium, cobalt, nickel, or any other magnetic metal in any form, whether liquid, solid, gel, foam, solid, or any other suitable form. The false eyelash and eyebrow hairs 54 attach to the magnetic formula within the cosmetic products described above with respect to FIGS. 6 and 7, without the use of adhesive, as seen in FIGS. 1 and 2. In some embodiments, the hairs 54 may be comprised of human or animal hair such as mink or any other animal hair. In other embodiments, the hairs 54 may be comprised of one or more types of fibers made from synthetic material(s) that include, but are not limited to nylon, polybutylene terephthalate, polyethylene terephthalate, recycled polyethylene terephthalate, and/or any other suitable material. In still other embodiments, the hairs 54 may be comprised of more than one of human hair, animal hair, and/or synthetic material. The individual false eyelash or eyebrow hairs 54 may have a smooth or rough surface texture, and each may taper to a fine point, though the length of taper may vary. A diameter of the hairs 54 may vary from between about 0.03 millimeters (mm) in diameter and about 0.25 mm in diameter, and the length of the hairs 54 may also vary from about 2 mm to about 30 mm. The color of the hairs 54 may vary as solid or multiple tones and the hairs may be offered in a variety of different sizes, shapes, colors, tones, etc. The hairs 54 may be attachable individually, as a cluster (in which case two or more hairs are glued or tied to a cotton or plastic fiber), as a substrate strip (in which case a larger number of hairs would be attached to a surface of a substrate in a generally perpendicular direction relative to a longitudinal axis of the substrate strip), or by some other technique that binds a large number of hairs together to maintain a false eyebrow or eyelash strip shape. The substrate strip may be a textile or non-woven material having a formula-facing or skin-facing surface and an outward facing surface, upon which the false hairs are applied.

The magnetization of the false hairs 54 (be they human, animal, or synthetic) may be achieved through several methods, as shown in FIG. 5. A first method 50 involves embedding, extruding, or otherwise infusing particles of a magnetic substance or substance with magnetic properties 56 into or onto each individual hair 54 with each hair 54 being embedded, extruded, or infused throughout the entire hair 54. In some embodiments, only a portion of the hair 54 may be embedded, extruded, or infused with the magnetic substance or substance with magnetic properties 56, for example, between about 10 percent and about 100 percent of the hair 54 may be embedded, extruded, or infused with the magnetic substance or substance with magnetic properties 56.

Another method 58 of magnetizing false hairs 54 involves coating each individual hair 54 with a substance with magnetic properties 60. Such coating may cover between about ten percent to about one hundred percent of a surface area of each hair 54. For example, a hair 54 may be coated only on the bottom third of the hair 54 so as to allow for attachment thereof to a bottom of a formula-treated natural lash 28 but also to facilitate more natural separation at the outermost tips of the lashes from the eyes of a user. Still another method 70 involves attaching a base 72 to an individual or cluster of hairs 54 that is itself a magnet or that is coated with a substance with magnetic properties from ten percent to one hundred percent of its surface area. In some embodiments, different hairs 54 may have a different percentage of coating. No matter the method of magnetization, each hair 54 is composed of, coated with, or attached to a material that is magnetic or attracted to a metal so as to engage the magnetic formula within each cosmetic product.

The attachment of false eyelash hairs 54 (be they human, animal, or synthetic) with magnetic properties to the magnetic formula of the corresponding cosmetic product may be achieved through several methods, as shown in FIGS. 1A and 1B. In accordance with FIG. 6, depending on whether the magnetic formula is applied by a mascara wand 30, a comb, or other applicator to the natural eyelashes 28 of the user, by a gel, cream, or liquid eye pencil/liner 12, 14 to the eyelid 36, 38 of the user, by an eyeshadow powder 16 to the eyelid 36, 38 of a user, or in any other suitable manner, the magnetic false eyelashes 54 may be applied directly to the natural eyelashes 28 of the user now coated in the magnetic formula within the cosmetic product, as seen in FIG. 1A, or to his or her eyelid now lined in the magnetic formula within the cosmetic product, as seen in FIG. 1B. Such application may be carried out by using a wand, tweezers, an eyelash curler, by hand, or by any other suitable applicator 100.

FIG. 2 shows the attachment of magnetized false eyebrow hairs 54 (be they human, animal, or synthetic) to the magnetic formula of the corresponding cosmetic product. As described above with respect to FIG. 7, regardless of whether the magnetic formula is applied by a brow mascara wand 18, 34, comb, or other applicator to the natural eyebrows 32 of a user, by a gel, cream, or liquid eyebrow pencil 20, 22 to the eyebrows 32 or brow bone skin 40 of a user, by an eyebrow powder 24, 44 to the eyebrow 32, 40 of a user, or in any other suitable manner, the magnetic false hairs 54 may be applied directly to the natural eyebrow 32 or brow bone skin 40 of the user by wand 102, tweezers, hand, or any other cosmetic applicator, as shown in FIG. 2.

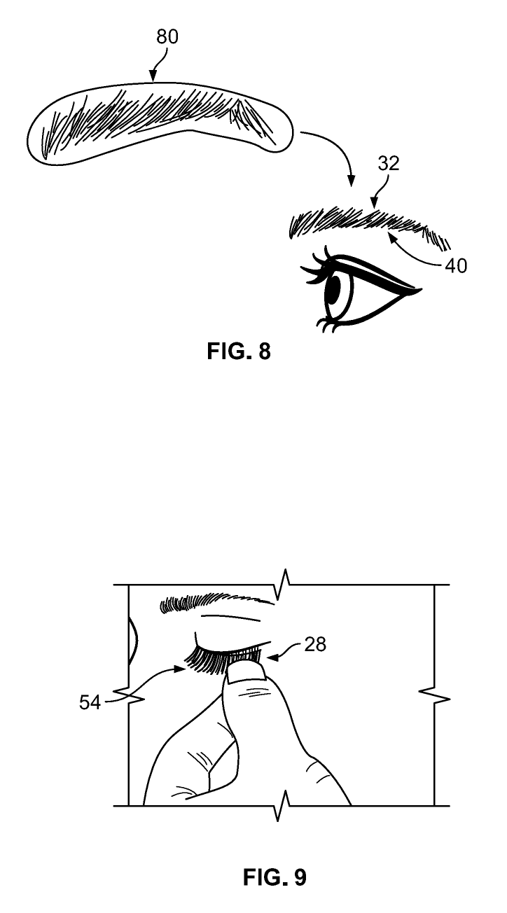

As seen in FIG. 8, a full, brow 80 with magnetic properties may be applied over a top of the natural eyebrow 32 or brow bone skin 40 of a user rather than attaching individual or clusters of the magnetized eyebrow hairs 54. The full magnetic brow 80 may be magnetized through the same methods depicted in FIG. 5 (i.e., infusion, coating of hair, coating of a magnetic material at an end of the hair, etc.). The hairs may be held together by threading, by a plastic strip, by glue, or by being melted together through heat or ultrasonic welding. Similarly, the full brow 80 with magnetic properties may be applied by the same methods as the individual eyebrow hairs 54 (i.e., by wand 102, tweezers, hand, or some other cosmetic applicator), as seen in FIG. 2. The brow 80 with magnetic properties applied by the methods disclosed herein retains flexibility and natural appearance of the brow, since there is no underlying adhesive to detract from the additional false eyebrow hairs.

Although the above embodiments describe the application of the magnetic formula in the cosmetic products as preceding the application of the false eyelashes or eyebrows, the reverse embodiment in which the eyelashes/eyebrows are put in place and then the formula applied over top of them (to the natural lashes, the eyelid, or the eyebrow) is equally feasible.

The magnetic elements may be formed by infusion into or addition to the formula or substance of a liquid, gel, cream and/or solid state via dipping, dying, soaking, molding, adhering with glue, and/or attaching via ultrasonic welding

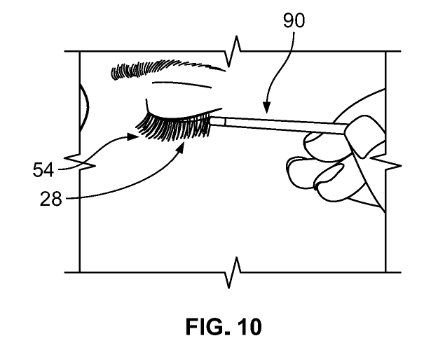

There are several alternatives for removing both the false eyelashes and eyebrows. As seen in FIG. 9, a user may gently slide the magnetic hairs away from natural eyelash and/or eyebrow hairs. Or, as seen in FIG. 10, the user may use a separate, stronger magnet 90 of the same magnetic attraction as the formula within the cosmetic product, which, when moved close to the applied false eyelashes and/or eyebrows, will draw the false hairs away from the natural hairs and onto the magnet. The removed hairs should retain their form and may be stored and reused.

The removal process for the magnetic formula in the cosmetic products may be removed using general makeup-removing techniques. Some of the cosmetic products may be waterproof formulas, which may be removed using general waterproof makeup-removing techniques.

All described products and variations thereof may be sold individually, or both the false eyelash system and the false eyebrow system may be sold as kits that contain one or more of the elements of the respective system. That is, a system kit may contain one or more of a cosmetic product with magnetic properties, false eyelash or eyebrow hairs with magnetic properties, and/or a magnet or removal substance or system for removing the false eyelash or eyebrow hairs. An example of a false eyelash kit may include a black mascara with magnetic properties, black eyelashes of a certain shape, size, length, and style, and an optional magnet for removal of the false eyelashes. In some embodiments, the kit may include a substance in liquid, gel, foam, solid, or any other format for removing the cosmetic product with magnetic formula. Each of those same products would also be available individually. There may be numerous kits that involve different variations of the type of cosmetic product, the color scheme of the cosmetic product, the color scheme of the false eyelashes or eyebrows, the shape of the false eyelashes or eyebrows, the size of the false eyelashes or eyebrows, the length of the false eyelashes or eyebrows, the style of the false eyelashes or eyebrows, and/or other cosmetic product variables (e.g., waterproof versus non-waterproof cosmetic products). In some embodiments, the kit may include false eyelashes or eyebrows of differing sizes, shapes, colors, etc.

While the principles of the present application have been described with respect to the application of false eyelash and eyebrow hairs, any of the embodiments described herein may be utilized for applying false hairs to any area of the body that lacks hair where desired or where a person would like more hair. For example, a user may desire to utilize one or more of the embodiments disclosed herein for use on the top of a head of the user, for a moustache or beard, a contoured hairline, or in any other desired location on the body. In some embodiments, the cosmetic products described herein may be clear and/or may not have any aesthetic features. For example, the cosmetic products may comprise the formula with magnetic properties and a component that allows for adhesion to the skin or hair.

1. A method of applying a false eyelash or eyebrow hair, the method comprising the steps of:
applying a cosmetic product to a natural eyelash, a natural eyebrow, an area around the natural eyelash, or an area around the natural eyebrow, the cosmetic product comprising magnetic properties; and
applying a false eyelash or eyebrow hair to the cosmetic product, at least a portion of the false eyelash or eyebrow hair comprising magnetic properties.

2. The method of claim 1, wherein the cosmetic product is selected from the group consisting of: an eyelash mascara, an eyebrow mascara, a gel or cream eyeliner pencil, a liquid or cream eyeliner, an eyeshadow powder, a gel or cream eyebrow pencil, a liquid or cream eyebrow pencil, or an eyebrow powder.

3. The method of claim 1, wherein the step of applying the cosmetic product includes applying the cosmetic product to the natural eyelash or natural eyebrow.

4. The method of claim 1, wherein the step of applying the cosmetic product includes applying the cosmetic product to skin of a user adjacent the natural eyelash or natural eyebrow.

5. The method of claim 1, wherein the false eyelash or eyebrow hair is infused, extruded, or embedded with a material that exhibits properties of magnetism or a metal attracted to magnets.

6. The method of claim 1, wherein the false eyelash or eyebrow hair is coated with a material that exhibits properties of magnetism or a metal attracted to magnets.

7. The method of claim 6, wherein between about 10 percent and about 100 percent of the false eyelash or eyebrow hair is coated with the material that exhibits properties of magnetism or the metal.

8. The method of claim 1, wherein the cosmetic product includes a metal selected from the group consisting of iron, magnesium, cobalt, and nickel.

9. The method of claim 1, wherein the cosmetic product includes a material that exhibits properties of magnetism.

10. The method of claim 1, wherein the cosmetic product comprises a material that exhibits properties of magnetism and the false eyelash or eyebrow hair comprises a material that exhibits properties of magnetism.

11. A system for applying a hair, the system comprising:
a cosmetic product comprising magnetic properties; and
a false hair comprising magnetic properties.

12. The system of claim 11, wherein the hair is infused, embedded, extruded, or coated with a magnetic substance or substance with magnetic properties.

13. The system of claim 12, wherein between about 10 percent and about 100 percent of the false hair is infused, embedded, extruded, or coated with the substance.

14. The system of claim 12, wherein only a bottom of the false hair is coated or infused with the magnetic substance or substance with magnetic properties.

15. The system of claim 11, wherein the cosmetic product is selected from the group consisting of: an eyelash mascara, an eyebrow mascara, a gel or cream eyeliner pencil, a liquid or cream eyeliner, an eyeshadow powder, a gel or cream eyebrow pencil, a liquid or cream eyebrow pencil, or an eyebrow powder.

16. The system of claim 11, wherein the cosmetic product includes a metal selected from the group consisting of iron, magnesium, cobalt, and nickel.

17. A kit for applying a false eyelash or eyebrow hair, the kit comprising:
a cosmetic product comprising magnetic properties;
an applicator for applying the cosmetic product; and
a false eyelash or eyebrow hair comprising magnetic properties.

18. The kit of claim 17, further including a magnet or a substance for removing the false eyelash or eyebrow hair.

19. The kit of claim 17, further including a substance for removing the cosmetic product.

20. The kit of claim 17, further including a plurality of false eyelash or eyebrow hairs, wherein at least two of the false eyelash or eyebrow hairs differ in size or color.

21. A kit for applying a false eyelash, the kit comprising:
a cosmetic product comprising a liquid-based eyeliner, the cosmetic product further comprising magnetized iron particles;
an applicator for applying the cosmetic product; and
a first false eyelash comprising a first substrate strip and a first plurality of hairs extending from the first substrate strip,
wherein the first substrate strip comprises a first plurality of magnets, which are magnetically attracted to the magnetized iron particles of the cosmetic product.

22. The kit of claim 21 further comprising instructions that direct a user to apply the cosmetic product to the skin of a user adjacent a natural eyelash.

23. The kit of claim 21 further comprising:
a second false eyelash comprising a second substrate strip and a second plurality of hairs extending from the second substrate strip,
wherein the second substrate strip comprises a second plurality of magnets, which are magnetically attracted to the magnetized iron particles of the cosmetic product.

24. A system for applying a hair, the system comprising:
a liquid-based eyeliner comprising a plurality of magnetized iron particles;
an applicator for applying the liquid-based eyeliner;
a first false eyelash, the first false eyelash comprising:
a first substrate strip;
a first plurality of false eyelashes extending from the first substrate strip; and
a first plurality of magnets provided along an underside of the substrate strip,
wherein the first plurality of magnets are magnetically attracted to the magnetized iron particles of the liquid-based eyeliner.

25. The system of claim 24 further comprising:
a second false eyelash, the second false eyelash comprising:
a second substrate strip;
a second plurality of false eyelashes extending from the substrate strip; and
a second plurality of magnets provided along an underside of the second substrate strip,
wherein the second plurality of magnets are magnetically attracted to the magnetized iron particles of the liquid-based eyeliner.
